(19) United States
(12) Patent Application Publication
(10) Pub. No.: US 2005/0085736 A1
(43) Pub. Date: Apr. 21, 2005

(54) Portable ECG detector

(76) Inventors: John A. Ambrose, Pleasantville, NY (US); John T. Coppola, Holmdel, NJ (US); Mary C. DeVoe, New York, NY (US)

Correspondence Address: JACKIE JAY SCHWARTZ, 1350 Broadway, Suite 1510, NEW YORK, NY, 10018, US

(21) Appl. No.: 10/688,637

(22) Filed: Oct. 17, 2003

(51) Int. Cl. A61B 5/0402

(52) U.S. Cl. 600/509

(57) ABSTRACT

An apparatus for detecting a myocardial infarction including a housing and a plurality of electrodes extending from the housing. Each of the plurality of electrodes is placed at a predetermined position on a body of a user for obtaining data representing bodily activity. A processor is positioned within the housing and connected to the plurality of electrodes. The processor analyzes the data obtained by the plurality of electrodes to form a baseline ECG and records the formed ECG. A means for storing the data representing the baseline ECG value is connected to the processor. The apparatus further includes a means for notifying a user of a myocardial infarction. Upon positioning the plurality of electrodes on the body of the user subsequently to storing the formed ECG, the plurality of electrodes obtain data representing current bodily activity. The processor then compares the current data to the stored. If upon determining the data representing current bodily activity deviates from the stored ECG by a predetermined deviation value, the processor controls the notification means to notify the user to seek medical attention.

### BACKGROUND OF THE INVENTION

1. Field of the Invention

The present invention relates generally to an electrocardiogram and, more specifically, to a portable ECG using an ST-segment analyzer for comparing a current ST-segment obtained from a user with a pre-stored baseline ST-segment obtained from the same user.

2. Description of the Prior Art

Coronary artery disease accounts for about 500,000 deaths per year in the United States. In addition there are around 1,000,000 heart attacks (myocardial infarctions) each year in the U.S. with substantial morbidity and overall mortality of about 4-12% in-hospital. It is also estimated that up to 50% of patents die from heart attacks before reaching the hospital. This is usually referred to as sudden cardiac death and is most commonly caused by an irregularity of the heart rhythm. While we now have effective therapy for the treatment of myocardial infarction including drugs or procedures to open up occluded coronary arteries and drugs to treat complication and prevent recurrences, one of the great public health difficulties with myocardial infarction is patient recognition of the significance of their symptoms.

Acute myocardial infarction is caused by an acute occlusion of a coronary artery that abruptly reduces or completely abolishes blood flow to a segment of the myocardium (heart muscle), this leads to the heart attack. In a case where there is a complete cessation of blood flow, it is imperative to open the artery as soon as possible. Effective therapy within 1-2 hours of the onset of the myocardial infarction may reduce the size of the heart attack by preserving muscle and reducing subsequent death and/or morbidity, congestive heart failure, serious cardiac arrhythmias and even a recurrent heart attack. Indeed, cardiologists and emergency medicine practitioners use a phrase that is repeated time and again, “time is muscle.”

One of the barriers to timely treatment of a heart attack is a patient's failure to recognize the importance of their symptoms, therefore delaying their call to 911 or their decision to go to an emergency room. Patients delay therapy for a number of reasons, most frequently is that the patient does not believe the symptoms are indicative of a heart attack. In this situation, patients may hesitate calling their doctor or seeking treatment and instead wait to see if the pain goes away on its own.

A diagnosis of myocardial infarction is made in a patient who, when presented with a typical history, has diagnostic changes on his or her electrocardiogram (ECG) of either ST segment elevation or, in some instances, ST segment depression. Additionally, elevations in certain substances in the blood may indicate acute damage to the heart muscle. However, this method of diagnosis is not the subject of the present application.

Numerous types electrocardiograms (ECGs) are known in the prior art. While these ECG's may be suitable for the purposes for which they were designed, they would not be as suitable for the purposes of the present invention, as hereinafter described.

### SUMMARY OF THE PRESENT INVENTION

The present invention relates generally to an electrocardiogram and, more specifically, to a portable ECG using an ST-segment analyzer for comparing a current ST-segment obtained from a user with a pre-stored baseline ST-segment obtained from the same user.

A primary object of the present invention is to overcome the shortcomings of prior art ECG devices by providing a portable ECG intended for home use as a diagnostic tool. The portable ECG of the present invention detects either ST segment elevation or ST segment depression and notifies the user of these changes.

Another object of the present invention is to utilize the portable ECG to alert a patient of the need to go directly to the nearest emergency room for an evaluation without delay. The ECG of the present invention takes an electrical picture of the heart and uses well-known and accepted standards based on changes in ST segment leads for diagnosing a heart attack. The portable ECG then presents this information to the user in a simple manner that allows the user to understand the information and react quickly to the information.

A further object of the present invention is to provide a portable ECG including an ST-segment analyzer for recording certain leads of the ECG when the user is not exhibiting any symptoms and stores them as a baseline value for subsequent analysis. When the user perceives a symptom and reapplies the device during the perceived symptom such that the leads are in a substantially similar position to that when taking the baseline reading, the same ECG leads are recorded and are compared to the baseline. Any deviation from the recorded value and the baseline value will be recognized and, if the deviation meets a predefined criteria, the user will be instructed to go immediately to the nearest emergency room.

An additional object of the present invention is to provide a portable ECG wherein predefined criteria are used in the analysis by the device to determine whether a user is exhibiting signs of a heart attack even in the absence of a pre-recorded baseline value.

Still another object of the present invention is to provide a portable ECG including an ECG lead system and processor positioned on a base. The ECG lead system records signals obtained through the leads, representing both baseline and current values. The processor analyzes the signals and recommends a plan of action such as directing the user to seek medical attention upon determining that the current value indicates a possible heart attack.

Yet a further object of the present invention is to provide a portable ECG wherein the baseline value is obtained by storing signals obtained through the ECG leads to record data for a period of 30 seconds when a user feels no discomfort. ST-segment signals received through leads 11, V2, and V5 corresponding to the inferior, anteroseptal and anterolateral areas of the heart respectively are recorded and stored for later comparison and analysis. Additionally, the user can selectively re-record the baseline value at any desired time. Preferably, the baseline is re-recorded at least once every six months.

Another object of the present invention is to provide a portable ECG able to be worn by a user wherein the placement of the ECG leads is guided by utilizing simple anatomic markers including the navel, the shoulders, and the hip bones. The navel is viewable through a clear window or alternatively, locatable via a finger hole for receiving a users finger for aligning the device with the navel of the user. The portable ECG includes at least five straps that are selectively extendable each having an electrode positioned at a first end thereof for contacting the shoulders and hips as well as areas to the left of the breast bone and under the left nipple. Signals representing the ECG data are received from the electrode on the end of the strap and recorded. The straps are preferably adjusted during the first usage of the wearable ECG when signals representing the baseline value are recorded in order to ensure proper placement when the user reapplies the portable ECG during a perceived symptom.

Yet a further object of the present invention is to provide a portable ECG wherein a user is lying down in a supine position having his raised at a 30° to 45° angle when the baseline value and any subsequent reading are obtained.

A further object of the present invention is to provide a portable ECG that uses a predetermined elevation of the ST segment of >1 millivolt and/or a ST segment depression of >1 millivolt from a baseline value in order to determine if the user should seek medical attention. Additionally, accurate diagnosis may be provided when a change in an ST-segment of only one lead is present.

Still another object of the present invention is to provide a portable ECG including a notification device for notifying a user that a sufficient elevation or depression in the ST-segment has been detected. The notification device is preferably at least one of an audible notification device or a visual notification device.

The foregoing and other objects and advantages will appear from the description to follow. In the description reference is made to the accompanying drawings, which forms a part hereof, and in which is shown by way of illustration specific embodiments in which the invention may be practiced. These embodiments will be described in sufficient detail to enable those skilled in the art to practice the invention, and it is to be understood that other embodiments may be utilized and that structural changes may be made without departing from the scope of the invention. In the accompanying drawings, like reference characters designate the same or similar parts throughout the several views.

The following detailed description is, therefore, not to be taken in a limiting sense, and the scope of the present invention is best defined by the appended claims.

### BRIEF DESCRIPTION OF THE DRAWING FIGURES

In order that the invention may be more fully understood, it will now be described, by way of example, with reference to the accompanying drawing in which:

FIG. 1 is front view of the portable ECG of the present invention configured to be worn by a user and having a plurality of selectively adjustable straps each having electrodes for recording an ECG signal;

FIG. 2 is a perspective view of the portable ECG of the present invention positioned on the body of a user;

FIG. 3 is a rear exploded view of the portable ECG of the present invention showing the connection of the electrodes to a processor;

FIG. 4 is a block diagram of the portable ECG of the present invention;

FIG. 5a is a graph of ECG reading showing a normal value;

FIG. 5b is a graph of an ECG reading showing the ST segment being elevated from its normal value;

FIG. 5c is a graph of an ECG reading showing the ST segment being depressed from its normal value;

FIG. 6 is a block diagram of the portable ECG of the present invention;

FIG. 7 is a front view of the portable ECG of the present invention being held in position on a user's body by securing straps;

FIG. 8 is an enlarged perspective view of an instruction panel of the portable ECG containing instructions on positioning the chest leads on a body of a user;

FIG. 9a is an enlarged perspective view of the left shoulder strap of the portable ECG of the present invention;

FIG. 9b is an enlarged perspective view of the right shoulder strap of the portable ECG of the present invention;

FIG. 9c is an enlarged perspective view of the right hip strap of the portable ECG of the present invention;

FIG. 9d is an enlarged perspective view of the left hip strap of the portable ECG of the present invention; and

FIG. 10 is a perspective view of the portable ECG of the present invention configured to be positioned atop a flat surface.

The foregoing and other objects and advantages will appear from the description to follow. In the description, reference is made to the accompanying drawing, which forms a part hereof, and in which is shown by way of illustration specific embodiments in which the invention may be practiced. These embodiments will be described in sufficient detail to those skilled in the art to practice the invention, and it is to be understood that other embodiments may be utilized and that structural changes may be made without departing from the scope of In the accompanying drawings, like reference characters designate the same or similar parts throughout the several views.

### DETAILED DESCRIPTION OF THE PREFERRED EMBODIMENTS

The following discussion describes in detail one embodiment of the invention and several variations of that embodiment. This discussion should not be construed, however, as limiting the invention to those particular embodiments. Practitioners skilled in the art will recognize numerous other embodiments as well. For a definition of the complete scope of the invention, the reader is directed to the appended claims.

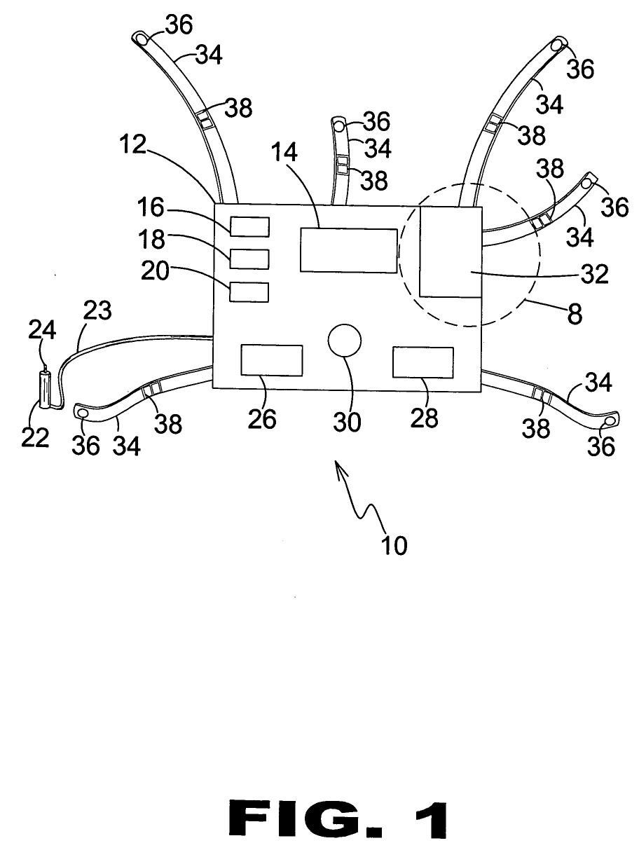

Turning now descriptively to the drawings, in which similar reference characters denote similar elements throughout the several views. FIGS. 1 through 10 illustrate a portable ECG of the present invention indicated generally by the numeral 10.

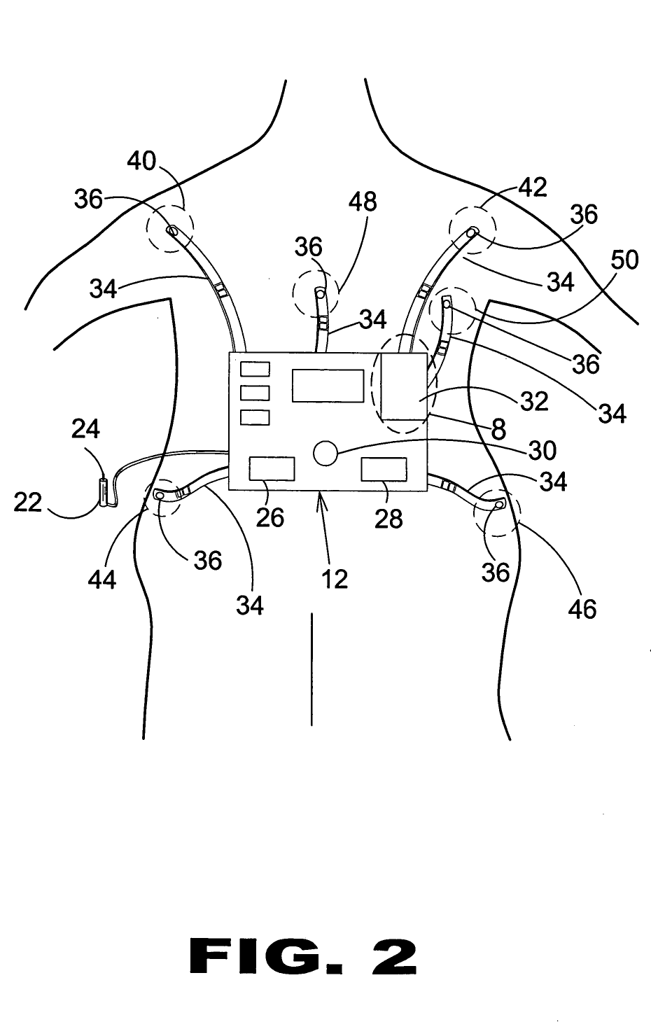

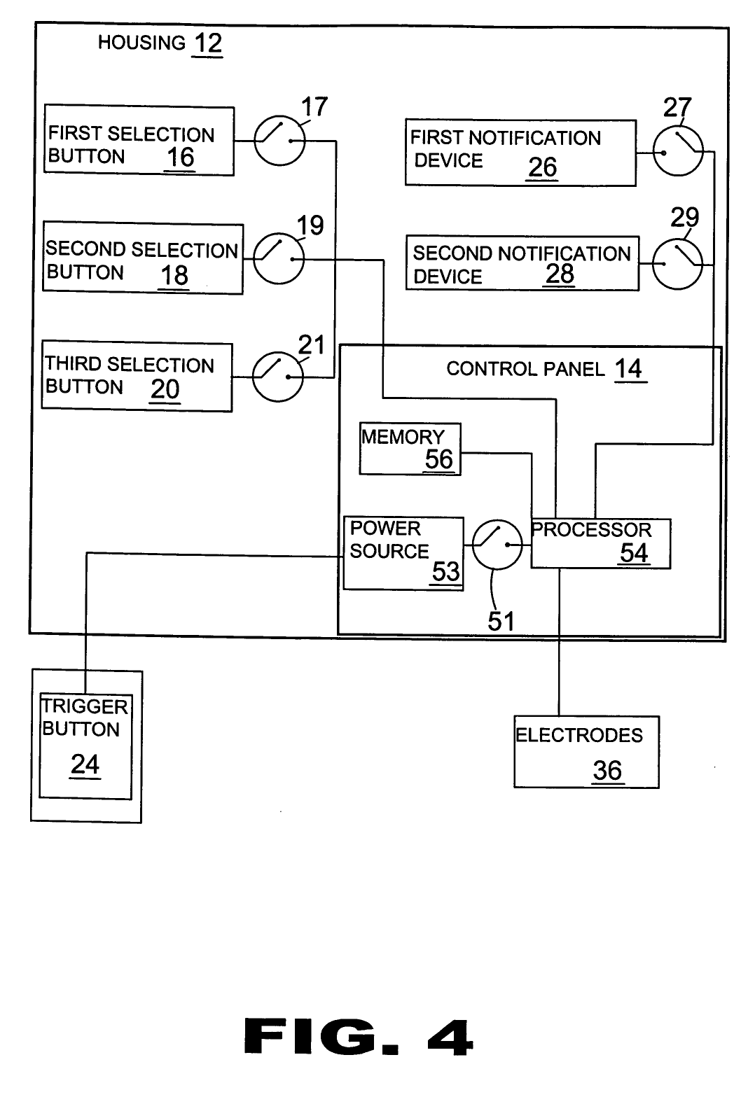

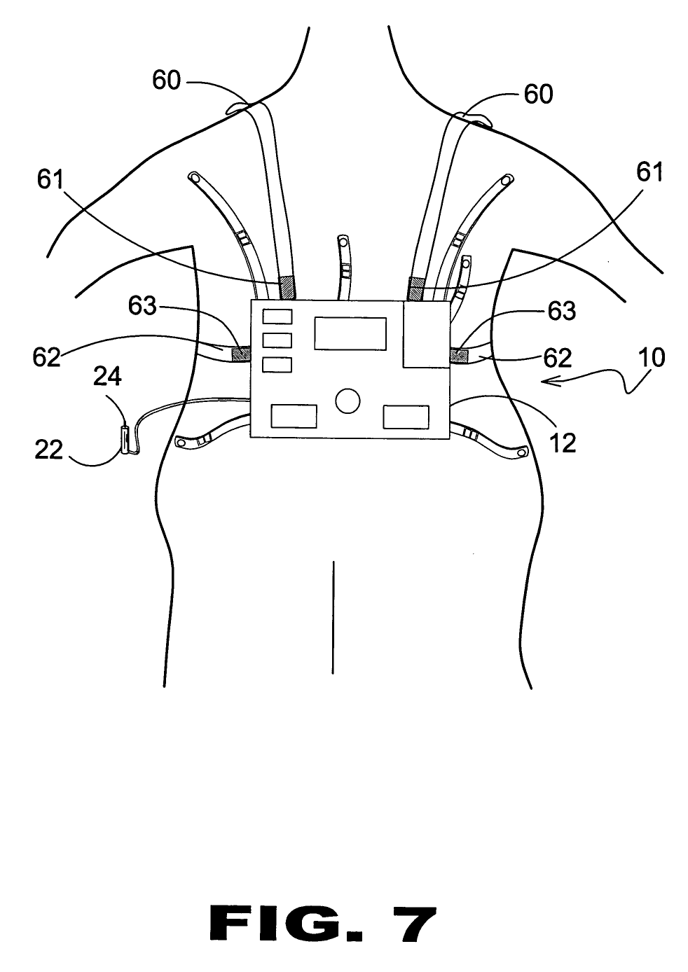

FIG. 1 is front view of a portable ECG 10 of the present invention. The portable ECG 10 includes a housing 12 which is selectively positioned on a body of a user as will be discussed hereinafter with specific reference to FIGS. 2 and 7. The housing 12 is preferably formed from lightweight pliable material so as to easily mold to the contours of the human body. The housing 12 includes a control panel 14 positioned on a first side thereof. The control panel 14 includes a processor 54 as shown in FIG. 4 for recording data representing an ECG baseline value and storing the data representing the baseline value in a memory. Additionally the processor 54 includes an ST-segment analyzer for comparing data representing a current ECG value with the data representing the pre-stored baseline value and determining if a deviation exists between the baseline and current values. The processor 54 also includes first and second notification devices 26 and 28 for notifying a user when a detected deviation indicates a possible myocardial infarction thereby instructing the user to seek medical attention.

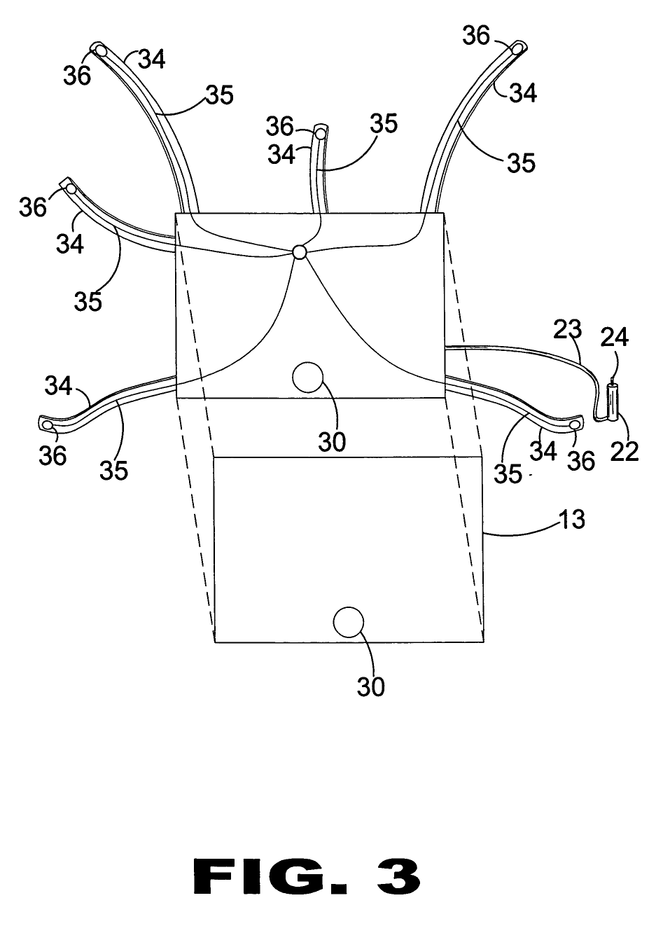

Extending from the housing 12 are a plurality of adjustable straps 34 each having an electrode 36 positioned at an end opposite a connection point between the strap 34 and the housing 12. Each electrode 36 is connected via connection wires 35 as shown in FIG. 3 to the processor 54 contained within the control panel 14. Each electrode 36 is provided to contact a predetermined positioned on the body of a user and obtain signals representing ECG values which are recorded by the processor 54. The positioning of the electrodes 36 will be discussed hereinafter with specific reference to FIGS. 2 and 7. Each strap 34 also includes an adjustment device 38 positioned thereon. The adjustment device 38 is preferably a slideable buckle for selectively elongating and reducing the length of each respective strap 34 thereby setting the placement of the electrodes 36 on the body of the user.

The portable ECG of the present invention is selectively operable using an operation unit 22. The operation unit 22 includes an operation trigger 24 and is connected to the processor 54 via an operation wire 23. The housing 12 also includes a first selection button 16, a second selection button 18 and a third selection button 20. The first selection button 16 is connected to the processor 54 for recording data representative of a baseline ST-segment value from the electrodes 36 positioned on the user. Additionally, the first selection button 16 allows a user to re-record a new baseline value at a later time. Perferably, a new baseline value is recorded at least every six (6) months. The second selection button 18 is connected to the processor 54 for recording data representative of a current ST-segment value from the electrodes 36 positioned on the user. Additionally, upon activation of the second selection button 18, the processor 54 compares the stored data representing the baseline ST-segment value with the data representing the current ST-segment. The third selection button 20 activates a diagnostic program which allows a user to determine if the portable ECG of the present invention is operating properly. The tasks associated with each of the selection buttons 16, 18 and 20 are described for purposes of example and any action can be assigned to any selection button 16, 18 and 20. Each task selected by depressing a selection button 16, 18, and 20 is performed upon activation of the trigger 24. Activating the tasks using the operation unit 22 allows the user to be in a comfortable position and allows the user to ensure that the portable ECG 10 is correctly positioned on their body. Preferably, the user is lying down in a supine position having their legs elevated at a 30° angle. Alternatively, the selection buttons 16, 18 and 20 can be used to control the activation of each task associated therewith thereby eliminating the need for the operation unit 22.

The housing 12 also includes a first notification device 26 and a second notification device 28. Preferably, the first and second notification devices 26 and 28, respectively, are visual notification devices such as a light which is selectively illuminable. It is also preferable that the first notification device 26 is illuminated using a color different that the color used to illuminate the second notification device 28. While the notification devices 26 and 28 are described as visual notification devices, the notifications devices 26 and 28 may also be audible notification devices whereby the sound emitted from the first notification device 26 is different from the sound emitted from the second notification device 28. Alternatively, the first and second notifications devices 26 and 28 may provide both a visual and audible notification. The first notification device 26 indicates when the processor 54 determines that the current ST-segment value is either elevated or depressed beyond a predetermined amount as compared to the baseline ST-segment value thus indicating a possible heart attack. Upon such a determination, the processor 54 causes the first notification device 26 to be illuminated thereby notifying the user that immediate medical attention should be sought. Should the processor 54 determine that the current ST-segment value is not elevated or depressed beyond a predetermined amount as compared to the baseline ST-segment value, the processor 54 causes the second notification device 28 to be illuminated. Additionally, when the user performs the diagnostic test function by using the third action button 20, and the diagnostic test is successful, the processor 54 causes both the first and second notification devices 26 and 28, respectively, to be illuminated simultaneously. 5.

FIG. 2 is a perspective view of the portable ECG 10 of the present invention positioned on the body of a user. The portable ECG 10 is positioned on the body of a user using simple anatomical markers to ensure proper placement thereof. The primary anatomical marker used for positioning of the portable ECG 10 is navel of the user. The housing 12 includes a navel window 30. Preferably, the navel window 30 is formed of a substantially transparent hypoallergenic material. The user places the housing 12 on his/her chest so that the user sees the navel through the navel window 30 on the housing 12. Alternatively, the housing 12 may include a navel hole for receiving a finger of a user therethrough for use in locating the user's navel. This is specifically helpful when the user is lying down in the supine position and desires to position the portable ECG on his/her body. Each strap 34 is then selectively adjusted so that the electrodes 36 at each end thereof contact specific areas on the user's body. A first electrode must contact the area substantially located within the circle labeled with the numeral 40 which represents the right shoulder. A second electrode must contact the area substantially located within the circle labeled with the numeral 42 which represents the left shoulder. A third electrode must contact the area substantially located within the circle labeled with the numeral 44 which represents the right hip and a fourth electrode must contact the area substantially located within the circle labeled with the numeral 46 which represents the left hip. The electrodes positioned in areas 40, 42, 44 and 46 are used for detecting limb lead ECG values. Additionally, a fifth electrode must contact the area substantially located within the circle labeled with the numeral 48 which represents an area just to the left of the breast bone and a sixth electrode must contact the area substantially located within the circle labeled with the numeral 50 representing an area just under the left nipple. The electrodes positioned in areas 48 and 50 are used for detecting precordial lead ECG values.

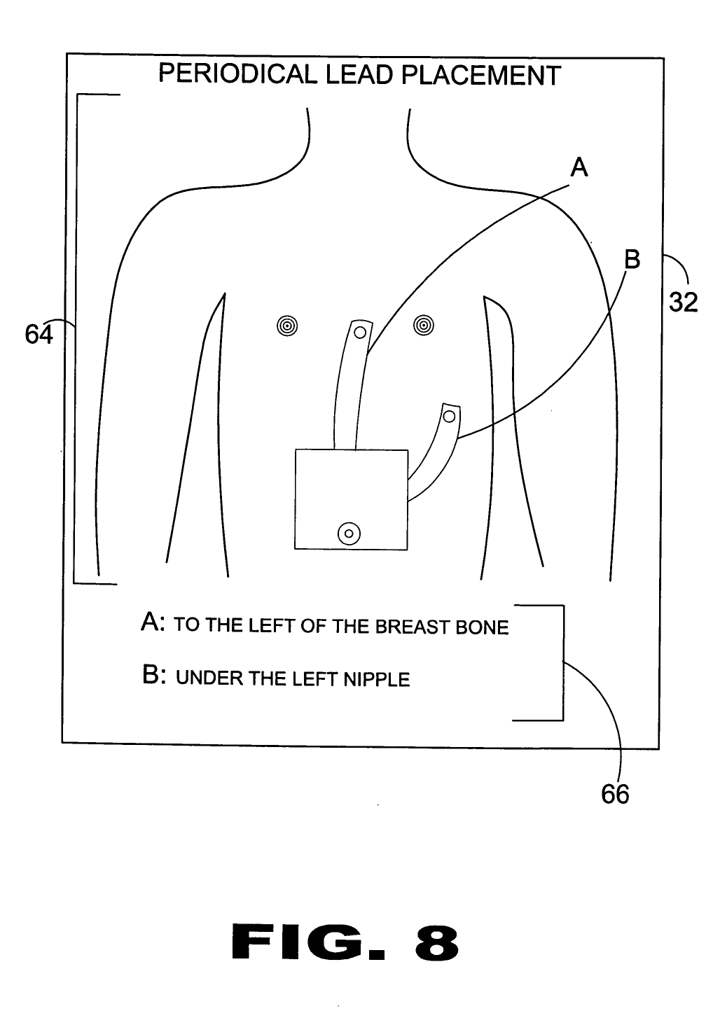

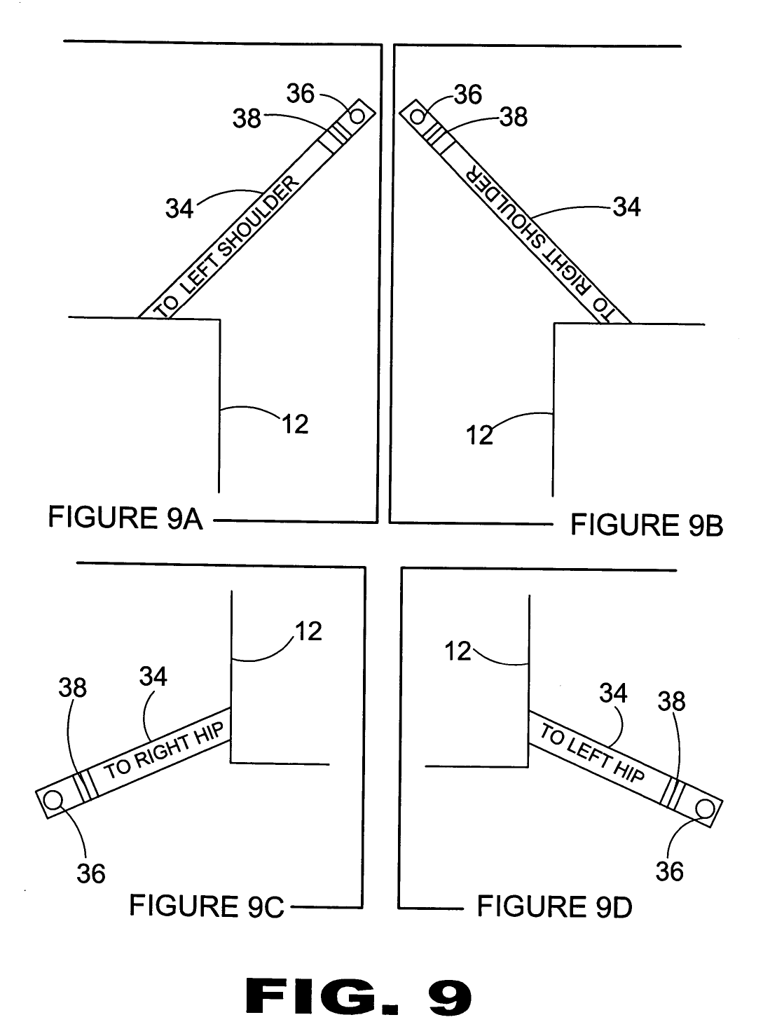

The portable ECG 10 includes instructions to notify the user of the specific location for each respective electrode 36 on each respective strap 34. As will be discussed hereinafter with specific reference to FIG. 9, each respective strap 34 includes instructions for proper electrode 36 placement. The instructions for the respective electrode 36 placement for the limb lead electrodes are located on each respective strap 34 as shown in FIG. 9. The housing 12 also includes an instruction panel 32 which is contained within the circled labeled with the numeral 8. The instruction panel 32 includes instructions on the proper placement of the precordial leads. The instruction panel 32 will be discussed in greater detail hereinafter with specific reference to FIG. 8.

By using the anatomical markers such as the navel, shoulders, hips, breast bone and left nipple, the portable ECG is easily positionable and reproduceable on the body of the user and readily ensures that a proper ECG reading can be obtained and used to determine whether or not the user should seek immediate medical assistance. The anatomical markers also ensure that the user will position the wearable ECG in substantially the same position on the body every time the wearable ECG is to be used.

FIG. 3 illustrates a rear exploded view of the portable ECG 10 of the present invention showing the connection of the electrodes 36 to the processor 54 (not shown in this Figure) through a recess 52. FIG. 3 shows a rear side of the housing 12 having a backing 13 that is detachably connected to the housing 12. The backing 13 includes a recess forming the navel window or hole 30. The rear side includes the recess 52 which opens into an inner section of the housing 12 as shown in FIG. 1. A connection wire 35 extends out from the recess 52 and along the rear side of the housing 12 and further along the length of a respective one of the adjustable straps 34. The connection wire 35 connects the electrode 36 positioned at the end of the adjustable strap 34 with the processor 54 contained within the housing 12. The number of connection wires 35 is equal to the number of electrodes 36 and the number of straps 34. When the backing 13 is secured to the rear side of the housing 12, the connection wires 35 are protected in order to ensure correct operation of the portable ECG by preventing any of the connection wires 35 from being detached from at least one of the processor 54 and the electrode 36.

FIG. 4 is a block diagram of the portable ECG of the present invention. The housing 12 includes the control panel 14 including the processor 54 having a power source 52 connected thereto via a power switch 51. A memory unit 56 is also positioned within the control panel 14 and connected to the processor 54. As discussed above with specific reference to FIG. 1, the first and second notification devices 26 and 28, respectively, are connected to the processor 54. A first notification switch is connected between the processor 54 and the first notification device 26 and a second notification switch 29 is connected between the second notification device and the processor 54. The first, second, and third selection buttons 16, 18 and 20, respectively, as well as the electrodes 36 are also connected to the processor 54. First, second and third selection switches 17, 19 and 21 are each connected between a respective one of the first, second and third selection buttons 16, 18 and 20 and the processor 54. Additionally, the trigger button 24 is connected to the power source 53 for selectively activating the portable ECG 10 of the present invention.

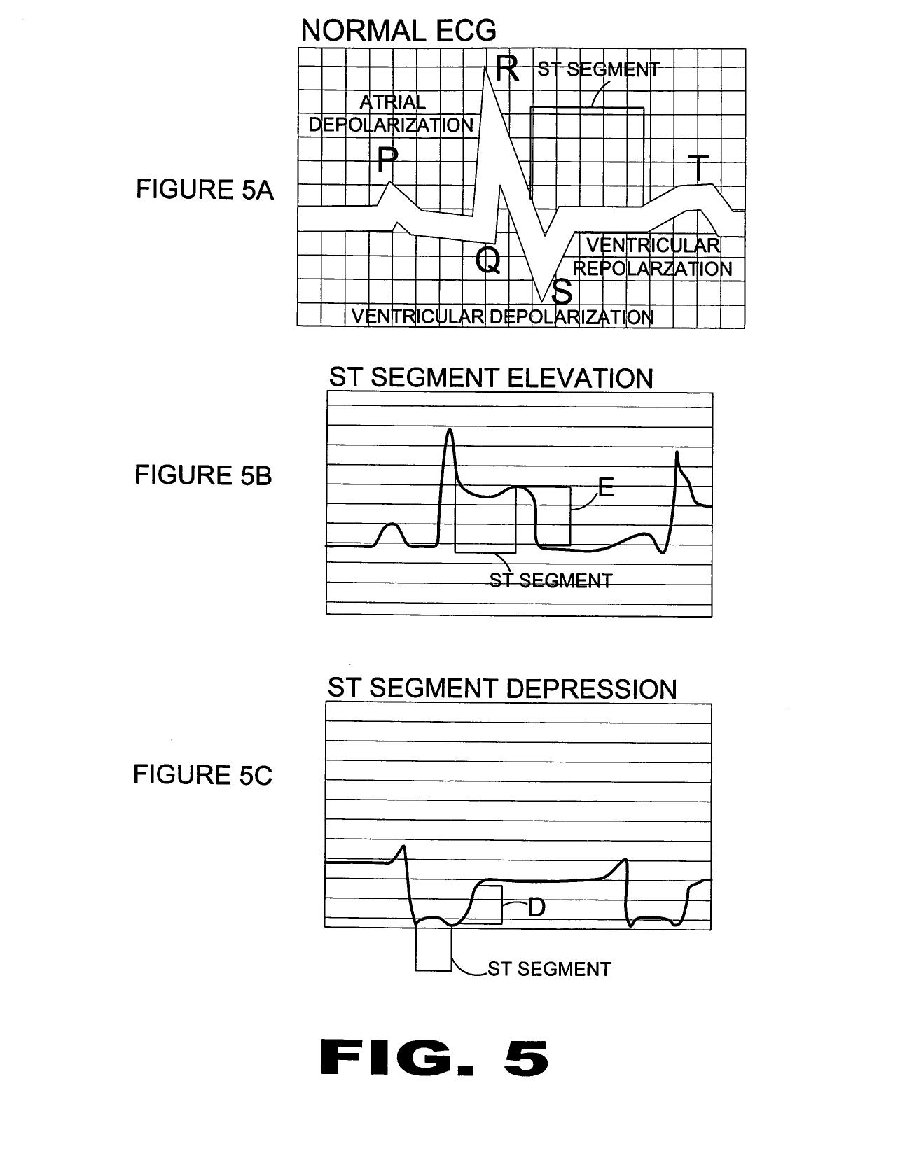

FIGS. 5a-5c are graphs showing different ECG readings. FIG. 5a shows a normal ECG reading taken from a person who is not experiencing any symptoms of a myocardial infarction. Shown in FIG. 5a, the ECG reading has an ST-segment that is normal and level. FIG. 5b shows an ECG reading where the ST-segment is elevated at level equal to the letter “E” and FIG. 5c shows an ECG reading where the ST-segment is depressed at a level equal to the letter “D”. If the value of elevation “E” as shown in FIG. 5b is greater than or equal to a predetermined value, e.g. 1 milivolt, then, the portable ECG 10 of the present invention will determine that the user is experiencing a myocardial infarction. Additionally, if the depression “D” is greater than or equal to a predetermined value, e.g. 1 milivolt, the portable ECG 10 will determine that the user is experiencing a myocardial infarction.

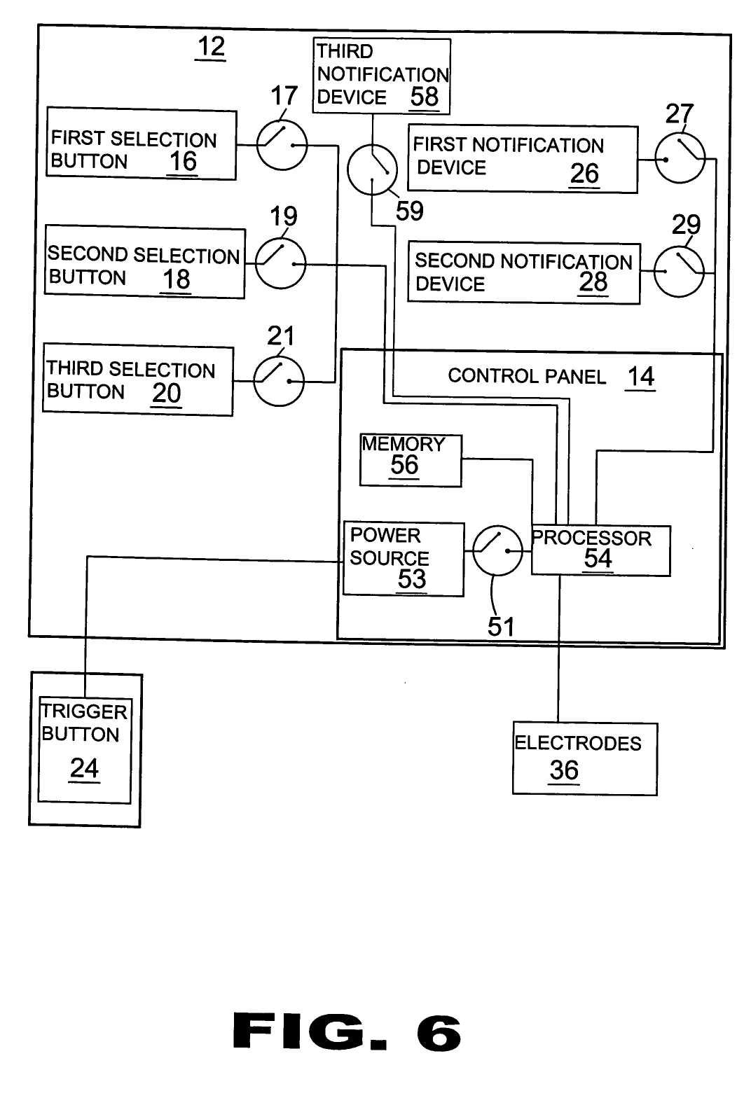

FIG. 6 is a block diagram of the portable ECG 10 of the present invention. The portable ECG 10 as shown in FIG. 6 includes all the elements as discussed hereinabove with respect to FIG. 4. Additionally, a third notification device 58 is connected to the processor 54 via a third notification switch 59. The third notification device 58 is preferably an audible notification device for providing an audible alert to the user, preferably when the portable ECG determines that the user should seek immediate medical attention.

FIG. 7 is a front view of the wearable ECG 10 of the present invention being held in position on a user's body. The wearable ECG 10 includes shoulder straps 60 and hip straps 62. The shoulder straps 60 are connected to the housing 12 via a connection device 61. The connection device 61 is preferably a releasable buckle for easy release. However, the connection device 61 may include any device that allows the shoulder straps 60 to be selectively detachable from the housing 12. This hip straps 62 are connected to the housing 12 via a second connection device 63. The second connection device 63 used to connect the hip straps 62 to the housing 12 is preferably the same as the first connection device 61. Upon securing the shoulder straps 60 and the hip straps 62 to the housing 12, the wearable ECG 10 of the present invention is releasably secured to the body of the user.

FIG. 8 is an enlarged perspective view of instruction panel 32 of the portable ECG 10 taken from within the circle labeled with the numeral 8 from FIG. 1. The instruction panel 32 preferable includes pictorial 64 and written instructions 66 for placing the precordial leads on the user's body. As discussed above with respect to FIG. 2, the precordial leads are recorded from two electrodes 36. One electrode 36 is placed to the left of the breast bone and a second electrode is placed under the left nipple. The instructions contained on the instruction panel 32 should clearly depict the placement of these electrodes to ensure that the user places the electrodes 36 in the correct positions. Preferably, the pictorial instructions 64 include a graphic display of a human body having the portable ECG 10 in the ideal position specifically showing the proper position of the precordial leads. As shown in the instructions, the user is preferably in the supine position with their legs elevated at an angle of substantially 30°. In addition to the pictorial display of instructions 64, the instruction panel 32 includes written instructions 66 for placement of the precordial leads on the user's body. Alternatively, instructions can be provided with the apparatus on a separate sheet and not positioned on the housing 12. For proper operation, the leads should be placed in substantially the same position during each use.

FIGS. 9a-9d are enlarged perspective views of the straps 34 of the portable ECG 10 of the present invention. FIG. 9a shows the left shoulder strap of the portable ECG 10 of the present invention. The left shoulder strap may include written instructions thereon directing the user to place the electrode 36 on the user's left shoulder. FIG. 9b shows the right shoulder strap of the portable ECG 10 of the present invention. The right shoulder strap may include written instructions thereon directing the user to place the electrode 36 on the user's right shoulder. FIG. 9c shows the right hip strap of the portable ECG 10 of the present invention. The right hip strap may include written instructions thereon directing the user to place the electrode 36 on the user's right hip. FIG. 9d shows the left hip strap of the portable ECG 10 of the present invention. The left hip strap may include written instructions thereon directing the user to place the electrode 36 on the user's left hip.

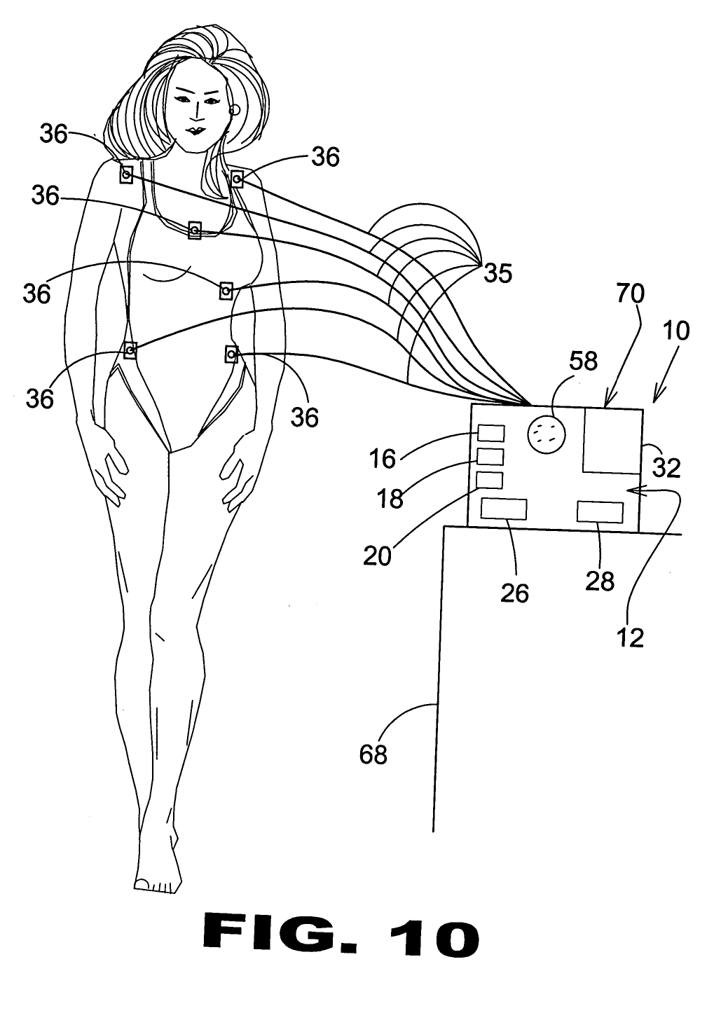

FIG. 10 is a perspective view of a table-top portable ECG 10 of the present invention. This embodiment is similar to the embodiment discussed with respect to FIGS. 7 and 9 but does not include shoulder straps for securing the housing 12 to the body of a user. The portable ECG 10 includes an external housing 70 that contains all circuits as discussed above with specific reference to FIGS. 4 and 6 therein. The control panel 14 is positioned on one face of the external housing 70. The control panel 12 includes the first selection button 16, the second selection button 18 and the third selection button 20. The control panel 14 also includes the first notification device 26, the second notification device 28 and the third notification device 58. Additionally, the control panel 12 includes the instruction panel 32 which contains all the instructions for correct placements of the electrodes 36 on the body of the user. A plurality of connection wires 35 extend from the external housing and each respective connection wire has an electrode connected at a first end thereof. The electrodes 36 are positioned on the body of the user in the same manner as discussed above with respect to FIG. 2. This embodiment allows a user to be in the most comfortable position when data representing the ECG signal is to be recorded by the electrodes 36.

The operation of the portable ECG 10 of the present invention will now be described with specific reference to the Figures. Preferably, the user is lying down in a supine position with the users legs elevated at approximately 30°. As discussed in FIG. 2, the user then places the housing 12 about the chest of the user using anatomical markers including the left and right shoulder, the left and right hips, the breast bone, left nipple and the navel. The housing 12 is then secured in position by the shoulder straps 60 and hip straps 62. The primary anatomical marker is the navel which is viewable through the optional navel window 30 extending through the control panel. Alternatively, the user may extend a finger through the navel hole for locating the navel for correctly positioning the housing 12 on the body of the user. Thereafter, limb leads are positioned in the correct position on the body of the user. The electrode on the left shoulder strap is positioned adjacent to the left shoulder. The electrode on the right shoulder strap is positioned adjacent to the right shoulder. The electrode on the left hip strap is positioned adjacent to the left hip. The electrode on the right hip strap is positioned adjacent to the right hip. The precordial leads are then positioned in the proper place on the body of the user. The electrode on the first precordial lead is positioned to the left of the user's breast bone and the electrode on the second precordial lead is positioned beneath the left nipple. Each respective strap is selectively adjustable so that the electrodes of the portable ECG 10 of the present invention are positioned in the optimal locations for recording ECG data values.

After the portable ECG 10 of the present invention is placed in the optimal position, the third selection button 20 is depressed causing the third selection switch 21 to move from a first open position to a second closed position. The third selection button 20 instructs the processor 54 to run a diagnostic program to determine if the portable ECG 10 is working properly. The diagnostic program is activated using the trigger 24 of the operation unit 22. Upon activating the trigger 24, the power switch 51 is caused to move from the first open position to the second closed position thereby completing an electrical circuit. The processor 54 then runs the diagnostic program which records sample readings from each electrode 36. If the processor 54 determines that the portable ECG 10 is working properly, the processor 54 causes the first notification switch 27 and the second notification switch 29 to move from the first open position to the second closed position. Thereafter, both the first and second notification devices are illuminated letting the user know that the portable ECG 10 of the present invention is working correctly.

Upon determining the portable ECG 10 is working properly, the user depresses the first selection button 16 which instructs the processor to analyze signals received from the plurality of electrodes to form and record a baseline ECG value. When the user depresses the first selection button 16, the first selection switch 17 is moved from a first open position to the second closed position. Then user depresses the trigger 24 of the operation unit 22 which causes the power switch 51 to move from the first open position to the second closed position thereby completing an electrical circuit. The processor 54 analyzes the signals received from the plurality of electrodes to form an ECG lasting for a period of at least thirty seconds. The recorded ECG data is then stored in the memory 56 as the baseline value. After the baseline value is recorded and stored, the first selection switch 17 and the power switch 51 are caused to move from the second closed position to the first open position therein disrupting the electrical circuit. After storing the baseline value, the user removes the portable ECG from his/her body for later use. At any later time, the user may re-record the baseline ECG value in order to store a new baseline ECG value by following the abovementioned steps and replacing the ECG data stored in the memory 56.

When a user perceives a symptom typically associated with a myocardial infarction, the user repositions the portable ECG 10 in the same manner as discussed above with specific reference to FIG. 2. In order to provide the best possible reading, the device should be used while symptoms are being experienced. At this time, the electrodes should be placed in substantially the same position as when the baseline value was taken and the user should be in substantially the same bodily position. The second selection button 18 is then depressed causing the second selection switch 19 to move from the first open position to the second closed position. The user depresses the trigger 24 of the operation unit 22 causing the power switch 51 to move from the first open position to the second closed position thereby completing an electrical circuit. Depressing the second selection button 18 instructs the processor 54 to analyze signals received from the plurality of electrodes to form and record a current ECG value. The current ECG value is then compared with the baseline ECG value which is stored in the memory 56. If the processor 54 detects an elevation or depression of greater than or equal to a predetermined value, e.g. 1 milivolt, of the ST-segment in the current ECG as compared to the ST-segment of the baseline ECG, the first notification switch 27 is caused to move from the first open position to the second closed position thereby causing the first notification device 26 to be illuminated. The first notification device 26 notifies the user that immediate medical attention should sought. Should the processor 54 not detect an elevation or depression of the ST-segment in the current ECG as compared to the ST-segment of the baseline ECG of greater than or equal to a predetermined value, e.g. 1 milivolt, the second notification switch 29 is caused to move from the first open position to the second closed position thereby causing the second notification device 28 to be illuminated for notifying the user that no troublesome deviation of ST-segments between the current and baseline values has been detected.

Additionally, if the portable ECG 10 includes a third notification device 58, and the processor 54 determines that an elevation or depression of the ST-segment in the current ECG as compared to the ST-segment of the baseline ECG of greater than or equal to a predetermined value, e.g. 1 milivolt, the third notification switch 59 is moved from the first open position to the second closed position. When the third notification switch 59 is in the closed position, the third notification device 58 emits an audible alert providing the user with an additional notification to ensure that the user seeks medical attention.

From the above description it can be seen that the present invention overcomes the shortcomings of the prior art by providing a portable ECG that utilizes simple anatomical markers to instruct a user in proper positioning of a plurality of electrodes on a user's body. Additionally, the portable ECG records a baseline ECG value and stores the baseline value in a memory and, upon a user perceiving symptoms of a myocardial infarction, the portable ECG records a current ECG value and compares the ST-segment of the current value with the ST-segment of the baseline values and notifies the user if immediate medical attention is needed.

It will be understood that each of the elements described above, or two or more together may also find a useful application in other types of methods differing from the type described above.

While certain novel features of this invention have been shown and described and are pointed out in the annexed claims, it is not intended to be limited to the details above, since it will be understood that various omissions, modifications, substitutions and changes in the forms and details of the device illustrated and in its operation can be made by those skilled in the art without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

1. An apparatus for detecting a myocardial infarction comprising:
a) a housing;
b) a plurality of electrodes extending from said housing, each of said plurality of electrodes being placed at a predetermined position on a body of a user for obtaining data representing bodily activity;
c) a processor positioned within said housing and connected to said plurality of electrodes, wherein said processor analyzes said data obtained by said plurality of electrodes to form a baseline ECG and records said formed ECG;
d) means connected to said processor for storing said data representing the baseline ECG value; and
e) means for notifying a user of a myocardial infarction, wherein upon positioning said plurality of electrodes on the body of the user subsequently to storing said formed ECG, said plurality of electrodes obtain data representing current bodily activity and said processor compares said data representing current bodily activity to said stored data, whereby if upon determining said data representing current bodily activity deviates from said stored ECG by a predetermined deviation value, said processor controls said notification means to notify the user to seek medical attention.

2. The apparatus as recited in claim 1, wherein said processor compares an ST-segment of said formed ECG with an ST-segment of said data representing current bodily activity.

3. The apparatus as recited in claim 2, wherein said predetermined deviation value is at least one of an elevation or depression of said ST-segment of said data representing current bodily activity of greater than or equal to 1 milivolt from said formed ECG.

4. The apparatus as recited in claim 1, further comprising means for activating said processor to analyze data obtained by said plurality of electrodes.

5. The apparatus as recited in claim 4, wherein said activating means is a hand-held operation unit having a trigger button positioned thereon, wherein said apparatus is activated by depressing said trigger button.

6. The apparatus as recited in claim 1, further comprising a second notification device for notifying a user that data representing current bodily activity does not deviate from said formed ECG by a predetermined amount.

7. The apparatus as recited in claim 6, wherein said first and second notification devices are visual alarms whereby said first notification device illuminates and is illuminated in a first color and said second notification devices illuminates and is illuminated in a second color different from said first color.

8. The apparatus as recited in claim 7, further comprising a third notification device, said third notification device is an audible notification device, and upon said processor determining said current ECG value differs from said baseline ECG value, said third notification device emits an audible alert in conjunction with said first notification device.

9. The apparatus as recited in claim 1, wherein said plurality of electrodes are positioned on the user's body using anatomical markers.

10. The apparatus as recited in claim 9, wherein said anatomical markers include a navel, a right shoulder, a left shoulder, a right hip, a left hip, a breast bone and a left nipple.

11. The apparatus as recited in claim 10, wherein said housing includes a navel window for viewing the user's navel therethrough and thereby aiding in positioning said apparatus on a body of the user.

12. The apparatus as recited in claim 11, further comprising a plurality of adjustable straps extending from said housing, each strap retaining a respective one of said plurality of electrodes.

13. The apparatus as recited in claim 12, wherein said plurality of adjustable straps is six adjustable straps, each respective adjustable strap includes an electrode positioned on a first end thereof, wherein upon positioning said control panel so that the user's navel is visible through said navel window:
a) a first electrode on a first strap is positioned adjacent to said left shoulder;
b) a second electrode on a second strap is positioned adjacent to said right shoulder;
c) a third electrode on a third strap is position adjacent to said left hip;
d) a fourth electrode on a fourth strap is positioned adjacent to said right hip;
e) a fifth electrode on a fifth strap is positioned left of said breast bone; and
f) a sixth electrode on a sixth strap is positioned beneath said left nipple.

14. The apparatus as recited in claim 1, further comprising:
a) a first selection button for instructing said processor to record and store data representing said formed ECG from said plurality of electrodes;
b) a second selection button for instructing said processor to record said data representing current bodily activity and compare said data representing current bodily activity with data representing said formed ECG; and
c) A third selection button for instructing said processor to run a diagnostic program for determining if said apparatus is functioning correctly.

15. The apparatus as recited in claim 9, wherein said housing includes a navel recess for receiving a user's finger therethrough thereby aiding in positioning said apparatus on a body of the user.

16. A method of determining if a user is experiencing a myocardial infarction using an apparatus comprising the steps of:
a) positioning a plurarlity of electrodes at predetermined positions on a body of a user;
b) activating the apparatus for a first time using an activation device;
c) recording data representing a baseline ECG value from the plurality of electrodes and storing said data in a memory unit;
d) removing the apparatus from the body of a user;
e) perceiving at least one symptom of a myocardial infarction;
f) repositioning the apparatus on the body of a user;
g) activating the apparatus for a second time using the activation device;
h) recording data representing current bodily activity;
i) comparing the data representing current bodily activity with the data representing the formed ECG for determining if the data representing current bodily activity deviates from the data representing the formed ECG by a predetermined deviation value; and
j) notifying a user, upon detecting the deviation that the symptoms are indicative of a myocardial infarction.

17. The method as recited in claim 16, wherein said step of comparing includes comparing an ST-segment of the data representing current bodily activity with an ST-segment of the data representing the formed ECG.

18. The method as recited in claim 16, further comprising the step of notifying a user that the data representing current bodily activity does not deviate from the data representing the formed ECG by a predetermined deviation value.

19. The method as recited in claim 16, wherein prior to each of said step of positioning and repositioning, further comprising the steps of lying down in a supine position and elevating the user's legs at an angle substantially equal to 30°.
